(19) United States
(12) Patent Application Publication
(10) Pub. No.: US 2025/0388956 A1
(43) Pub. Date: Dec. 25, 2025

(54) COMPOSITIONS AND METHODS FOR IDENTIFICATION OF CHROMOSOMAL MICRODELETIONS

(71) Applicant: Natera, Inc., San Carlos, CA (US)

(72) Inventors: Hemant Pawar, Foster City, CA (US); Wenbo XU, San Carlos, CA (US); Dianne Keen-Kim, Hillsborough, CA (US); Maxim BREVNOV, Union City, CA (US)

(73) Assignee: Natera, Inc., San Carlos, CA (US)

(21) Appl. No.: 18/877,708

(22) PCT Filed: Jun. 28, 2023
(86) PCT No.: PCT/US2023/026471

§ 371 Date: Dec. 20, 2024

Related U.S. Application Data
(60) Provisional application No. 63/356,912, filed on Jun. 29, 2022.

(51) Int. Cl. C12Q 1/686; C12Q 1/48; C12Q 1/6806; C12Q 1/6809; C12Q 1/6869; C12Q 1/6886

(52) U.S. Cl. CPC C12Q 1/686; C12Q 1/48; C12Q 1/6806; C12Q 1/6809; C12Q 1/6869; C12Q 1/6886; C12Y 207/07; C12Q 2600/156; C12Q 2600/166

(57) ABSTRACT

Provided herein are compositions to be used as a positive control for detection of one or more microdeletions of interest in a sample. The positive control can be used to determine an error and an efficiency rate for assays used to identify microdeletions such as 22q1 1.2 deletion (DiGeorge syndrome), chromosome 5p15.2 (Cri-du-chat), 1p36 deletion, 15q1 1.2˜q13 deletion (Prader-Willi syndrome), and/or 15q1 1˜q13 (Angelman syndrome).

### BACKGROUND

Subchromosomal abnormalities such as microdeletions and duplications may result in severe physical and/or intellectual impairments. Eight of the microdeletion syndromes have an aggregate incidence of more than 1 in 1000, making them nearly as common as fetal autosomal trisomies.

In younger women, the risk for a clinically significant microdeletions exceeds the risk for Down syndrome. Because some infants with subchromosomal abnormalities may benefit from early therapeutic intervention prenatal detection is important for optimal management.

Subchromosomal abnormalities such as microdeletions or duplications are relatively harder to detect than chromosomal abnormalities because of their small size. There is a need for improved methods and compositions for detecting subchromosomal abnormalities such as microdeletions.

### SUMMARY OF THE INVENTION

The present disclosure provides positive control compositions for detection of microdeletions.

The present disclosure provides a composition comprising an engineered nucleic acid construct for use as a positive control for detection of one or more microdeletions of interest in a sample, wherein the construct is engineered from a reference nucleic acid sequence comprising a microdeletion of interest, wherein the construct comprises a 5′ end region, a central region, and a 3′ end region, wherein the 5′ end region and 3′ end region comprise reference sequences flanking the microdeletion of interest, wherein the central region of the construct is a DNA barcode, and wherein the barcode replaces the microdeletion sequence of the reference nucleic acid sequence.

In some embodiments, the microdeletion of interest corresponds to 22q11.2 deletion, 5p15.2 deletion, 1p36 deletion, 15q11.2-q13 deletion, or 15q11-q13 deletion. In some embodiments, the microdeletion of interest is associated with a cancer.

In some embodiments, the composition comprises (i) a first engineered nucleic acid construct comprising a barcode replacing a microdeletion of interest corresponding to 22q11.2 deletion, (ii) a second engineered nucleic acid construct comprising a barcode replacing a microdeletion of interest corresponding to 5p15.2 deletion, (iii) a third engineered nucleic acid construct comprising a barcode replacing a microdeletion of interest corresponding to 1p36 deletion, (iv) a fourth engineered nucleic acid construct comprising a barcode replacing a microdeletion of interest corresponding to 15q11.2-q13 deletion, and (v) a fifth engineered nucleic acid construct comprising a barcode replacing a microdeletion of interest corresponding to 15q11-q13 deletion.

In some embodiments, the barcode is from about 6 base pairs to about 9 base pairs.

In some embodiments, the 5′ and 3′ end regions of the construct comprise at least one single nucleotide polymorphism (SNP) of interest.

In some embodiments, the 5′ and 3′ end regions of the construct comprise sequences recognized by primers that target a SNP within or flanking the microdeletion of interest.

In some embodiments, the reference nucleic acid sequence is maternal DNA, and wherein the SNP of interest is changed to allow the construct to act as the positive control for a child DNA.

In some embodiments, the size of the construct is from about 100 bp to about 200 bp, or from about 160 bp to about 200 bp.

In another aspect, the present disclosure provides a method of preparing the construct for use as a positive control for detection of microdeletions of interest in a sample, wherein the method comprises obtaining a reference nucleic acid; isolating a nucleic acid sequence comprising a 5′ end and 3′ end flanks a microdeletion of interest; and replacing the central region of the reference nucleic acid sequence corresponding to the microdeletion of interest with a barcode.

In some embodiments, the reference nucleic acid is obtained from a cell line suitable for use as a positive control for detecting the one or more microdeletions.

In some embodiments, the reference nucleic acid is mono-nucleosomal. In some embodiments the reference nucleic acid is genomic DNA.

In another aspect, the present disclosure relates to a method of preparing a preparation of amplified DNA derived from a sample or a fraction thereof useful for identifying one or more microdeletions associated with a disease or disorder, comprising: (a) preparing a construct for use as a positive control for detection of one or more microdeletions; (b) adding the construct from (a) into the sample or fraction thereof to obtain a spiked sample and extracting nucleic acids from the spiked sample or fraction thereof; (c) performing targeted amplification on the spiked sample or fractions thereof from (b) to amplify one or more target regions comprising microdeletions of interests to obtain amplicons; and (d) analyzing the amplicons or portions thereof from (c) to determine (i) whether the amplicons comprises the amplified construct as a positive control, and (ii) whether the amplicons comprises the one or more microdeletions of interest.

In some embodiments, the preparing a construct for use as a positive control for detection of one or more microdeletions is performed by chemical synthesis and subsequent PCR amplification of the synthesized construct.

In some embodiments, the sample is a plasma sample and comprises cell-free DNA. In some embodiments, the plasma sample comprises maternal and fetal cell-free DNA, and wherein a SNP in the construct is changed to act as a positive control for the fetal cell-free DNA. In some embodiments, the sample comprises circulating tumor DNA (ctDNA).

In some embodiments, at least 5 microdeletions of interest are amplified in a single reaction volume, and wherein a construct for use as a positive control is prepared for each of the at least 5 microdeletions of interest. In some embodiments, the herein disclosed method of preparing a preparation of amplified DNA derived from a sample or a fraction thereof useful for identifying one or more microdeletions associated with a disease or disorder, and the one or more microdeletions comprise 22q11.2 deletion (DiGeorge syndrome), chromosome 5p15.2 (Cri-du-chat), 1p36 deletion, 15q11.2˜q13 deletion (Prader-Willi syndrome), and/or 15q11˜q13 (Angelman syndrome).

In some embodiments, the herein disclosed method of preparing a preparation of amplified DNA derived from a sample or a fraction thereof useful for identifying one or more microdeletions associated with a disease or disorder further comprises sequencing to detect (i) the presence of the construct as a positive control, and (ii) the presence of the one or more microdeletions of interest.

In some embodiments, the herein disclosed method of preparing a preparation of amplified DNA derived from a sample or a fraction thereof useful for identifying one or more microdeletions associated with a disease or disorder an efficiency and an error rate is determined for each amplification reaction by using the positive control, wherein the efficiency and the error rate is used to determine the presence of the one or more microdeletions of interest.

In some embodiments, the present disclosure provides an amount of the construct to be added to the sample is determined by (a) mixing DNA from normal female cell line and the construct in a range of proportions to generate a titration series to determine the Limit of Detection; (b) adding the mixture from (a) to DNA depleted plasma; (c) perform targeted amplification of the microdeletion that the construct is positive control for; and (d) determination of the proportion of the construct and the mono-nucleosomal DNA from the normal cell line that allows detection of the construct. In some embodiments, the DNA is mononucleosomal or genomic DNA.

In another aspect, the present disclosure provides a method of preparing a sample comprising nucleic acids, comprising spiking a sample with the composition. In some embodiments, the sample is a plasma sample from a mother.

In some embodiments, the detection of SNPs flanking the barcodes that replace the microdeletion in the engineered construct in the amplicons demonstrates that the assays for detecting SNPs flanking and within the microdeletion work, thereby confirming that the engineered constructs can be used as the positive control.

### BRIEF DESCRIPTION OF THE DRAWINGS

FIG. 1 is a graphical depiction of an engineered nucleic acid construct for use as a positive control for detection of microdeletions. MD is short for microdeletion. STAR primers refer to the STAR PCR protocol described elsewhere herein. STAR is short for Specific Target Amplification Reaction.

FIG. 2 is a flowchart depicting the use of the herein disclosed engineered nucleic acid construct for use as a positive control for detection of microdeletions.

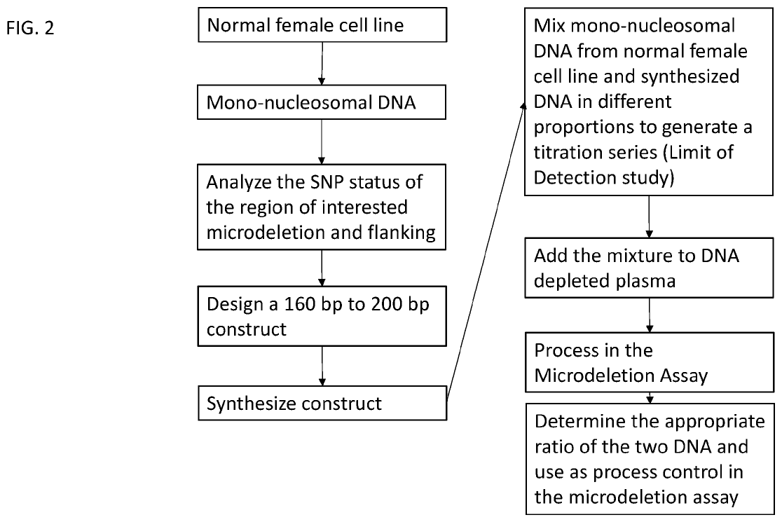

### DETAILED DESCRIPTION

Reference will now be made in detail to some specific embodiments of the invention contemplated by the inventors for carrying out the invention. Certain examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included.

The present disclosure provides improved methods for determining microdeletions or other subchromosomal abnormalities of interest. In some embodiments, the present disclosure provides methods for non-invasive prenatal testing (NIPT), specifically, identifying microdeletions or other subchromosomal abnormalities in a fetus by performing targeted amplification and using the herein disclosed engineered positive control construct corresponding to the microdeletion of interest.

In particular, the present disclosure provides a composition comprising an engineered nucleic acid construct for use as a positive control for detection of one or more microdeletions of interest in a sample, wherein the construct is engineered from a reference nucleic acid sequence comprising a microdeletion of interest, wherein the construct comprises a 5′ end region, a central region, and a 3′ end region, wherein the 5′ end region and 3′ end region comprise reference sequences flanking the microdeletion of interest, and wherein the central region of the construct is a DNA barcode, wherein the barcode replaces the microdeletion sequence of the reference nucleic acid sequence.

The barcode is unique to a particular microdeletion region so that the PCR of the positive control for a particular microdeletion PCR assay can be determined and analyzed. The PCR of the positive control can be used to determine the efficiency and error rate of the PCR assay for a microdeletion of interest, thereby improving the accuracy of the microdeletion test.

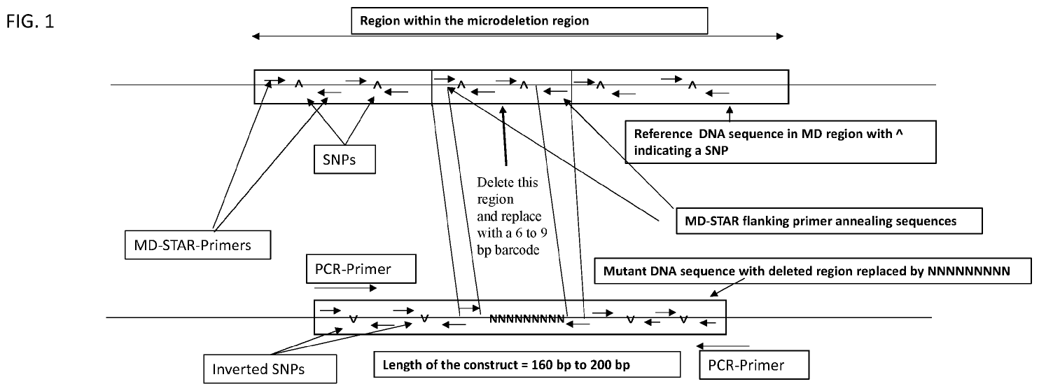

A graphical depiction of an exemplary engineered nucleic acid construct for use as a positive control for detection of one or more microdeletions of interest as disclosed herein is provided in FIG. 1. As shown in FIG. 1, the engineered nucleic acid construct comprises a barcode that replaces the region corresponding to a microdeletion of interest and retains the 5′ end region and 3′ end region that flank the region corresponding to the microdeletion of interest. In some embodiments, this DNA barcode may be about 4 to about 10 base pairs, about 5 to about 10 base pairs, 6 to about 10 base pairs, 6 to about 9 base pairs, 6 to about 9 base pairs, about 6 to about 12 base pairs, about 6 to about 15 base pairs, about 6 to about 18 base pairs, about 6 to about 20 base pairs, about 6 base pairs, about 7 base pairs, about 8 base pairs, or about 10 base pairs.

In some embodiments, the size of the construct is from about 100 bp to about 200 bp, from about 160 bp to about 200 bp, is from about 100 bp to about 300 bp, from about 100 bp to about 400 bp, or from about 100 bp to about 500 bp.

In some embodiments, the microdeletion of interest corresponds to 22q11.2 deletion, 5p15.2 deletion, 1p36 deletion, 15q11.2-q13 deletion, or 15q11-q13 deletion. The 22q11.2 deletion is associated with DiGeorge syndrome. In some embodiments, the microdeletion is associated with Prader-Willi syndrome. In some embodiments, the microdeletion is associated with Angelman syndrome. In some embodiments, the microdeletion is 1p36 deletion. In some embodiments the microdeletion is associated with the Cri-du-chat syndrome.

In some embodiments, the composition comprises (i) a first engineered nucleic acid construct comprising a barcode replacing a microdeletion of interest corresponding to 22q11.2 deletion, (ii) a second engineered nucleic acid construct comprising a barcode replacing a microdeletion of interest corresponding to 5p15.2 deletion, (iii) a third engineered nucleic acid construct comprising a barcode replacing a microdeletion of interest corresponding to 1p36 deletion, (iv) a fourth engineered nucleic acid construct comprising a barcode replacing a microdeletion of interest corresponding to 15q11.2-q13 deletion, and (v) a fifth engineered nucleic acid construct comprising a barcode replacing a microdeletion of interest corresponding to 15q11-q13 deletion. In some embodiments, the composition comprises a plurality of engineered nucleic acid constructs, wherein each construct is a positive control for a microdeletion region of interest. The composition may comprise 2, 3, 4, 5, 6, 7, 8, 9, 10, 11, 12, 15, 20, 25 or 50 different engineered nucleic acid constructs on the low end and 3, 4, 5, 6, 7, 8, 9, 10, 11, 12, 15, 20, 25, 50, 100 or 250 different engineered nucleic acid constructs on the high end, wherein each construct is a positive control for a microdeletion region of interest.

In some embodiments, the 5′ and 3′ end regions of the construct comprise at least one single nucleotide polymorphism (SNP) of interest. In some embodiments, the 5′ and 3′ end regions of the construct comprise sequences recognized by primers that target a SNP within or flanking the microdeletion of interest. In some embodiments, the reference nucleic acid sequence is maternal DNA, and wherein the SNP of interest is changed (or inverted) to allow the construct to act as the positive control for a child DNA.

The term “single nucleotide polymorphism (SNP)” refers to a single nucleotide that may differ between the genomes of two members of the same species. The usage of the term should not imply any limit on the frequency with which each variant occurs.

The term “sequence” refers to a DNA sequence or a genetic sequence. It may refer to the primary, physical structure of the DNA molecule or strand in an individual. It may refer to the sequence of nucleotides found in that DNA molecule, or the complementary strand to the DNA molecule. It may refer to the information contained in the DNA molecule as its representation in silico.

The term “locus” refers to a particular region of interest on the DNA of an individual, which may refer to a SNP, the site of a possible insertion or deletion, chromosome or portion thereof, or the site of some other relevant genetic variation. Disease-linked SNPs may also refer to disease-linked loci.

The term “polymorphic allele” or “polymorphic locus” refers to an allele or locus where the genotype varies between individuals within a given species. Some examples of polymorphic alleles include single nucleotide polymorphisms, short tandem repeats, deletions, duplications, and inversions.

In another aspect, the present disclosure provides a method of preparing the construct for use as a positive control for detection of microdeletions of interest in a sample according to claims 1-8, wherein the method comprises obtaining a reference nucleic acid; isolating a nucleic acid sequence comprising a 5′ end and 3′ end flanks a microdeletion of interest; and replacing the central region of the reference nucleic acid sequence corresponding to the microdeletion of interest with a barcode.

As used herein, the term “nucleic acid” refers to nucleic acids in its broadest meaning and is not limited to any particular form or type of nucleic acid. In some embodiments, nucleic acids may genomic DNA, mono-nucleosomal, di-nucleosomal, tri-nucleosomal, cell-free DNA, or DNA obtained from cells, tissues, or organs. Nucleic acids may also refer to RNA of any kind, including without limitation small non-coding RNAs such as miRNA, tRNA, or piwiRNA. The term nucleic acids may also include synthetic or modified nucleic acids.

In some embodiments, the reference nucleic acid is obtained from a cell line suitable for use as a positive control for detecting the one or more microdeletions.

In some embodiments, the reference nucleic acid is mono-nucleosomal.

In another aspect, the present disclosure relates to a method of preparing a preparation of amplified DNA derived from a sample or a fraction thereof useful for identifying one or more microdeletions associated with a disease or disorder, comprising: (a) preparing a construct for use as a positive control for detection of one or more microdeletions; (b) adding the construct from (a) into the sample or fraction thereof to obtain a spiked sample and extracting nucleic acids from the spiked sample or fraction thereof; (c) performing targeted amplification on the spiked sample or fractions thereof from (b) to amplify one or more target regions comprising microdeletions of interests to obtain amplicons; and (d) analyzing the amplicons or portions thereof from (c) to determine (i) whether the amplicons comprises the amplified construct as a positive control, and (ii) whether the amplicons comprises the one or more microdeletions of interest.

In some embodiments, the construct for use as a positive control for detection of one or more microdeletions is prepared by chemical synthesis and subsequent PCR amplification.

In some embodiments, the biological sample is a blood, plasma, serum, or urine sample. In some embodiments, the sample is a plasma sample and comprises cell-free DNA. In some embodiments, the sample comprises any fragment or segment of genomic DNA. In some embodiments, the sample comprises cellular DNA. As used herein, “cellular DNA” refers to DNA obtained from cells, organs, and tissues.

As used herein, the term “cell-free DNA” or “cfDNA” refers to DNA that is free-floating in biological samples. In some embodiments, the biological sample is a blood, plasma, serum, or urine sample. In some embodiments, the sample is a plasma sample and comprises cell-free DNA. In some embodiments, the biological sample is from a pregnant mother. In some embodiments, the isolated cfDNA is a mixture of fetal and maternal cfDNA. In some embodiments, the plasma sample comprises maternal and fetal cell-free DNA, and wherein a SNP in the construct is changed to act as a positive control for the fetal cell-free DNA.

In some embodiments, the biological sample comprises circulating tumor DNA (ctDNA). In some embodiments, the compositions herein are used as positive control for detecting ctDNA, and detecting or monitoring a cancer or tumor. ctDNA has been found in the circulation of patients diagnosed with malignancies including but not limited to lung cancer, prostate cancer, colon, and breast cancer. Identification of genomic instabilities associated with cancers that can be determined in the ctDNA in cancer patients is a potential diagnostic and prognostic tool. In one embodiment, the method of the invention assesses microdeletions of interest in a sample comprising a mixture of nucleic acids derived from a subject that is suspected or is known to have cancer e.g. carcinoma, sarcoma, lymphoma, leukemia, germ cell tumors and blastoma. In one embodiment, the sample is a plasma sample derived (processes) from peripheral blood and that comprises a mixture of cfDNA derived from normal and cancerous cells (ctDNA). In another embodiment, the biological sample that is needed to determine whether a microdeletion is present is derived from a mixture of cancerous and non-cancerous cells from other biological fluids including but not limited to serum, sweat, tears, sputum, urine, sputum, ear flow, lymph, saliva, cerebrospinal fluid, bone marrow suspension, vaginal flow, transcervical lavage, brain fluid, ascites, milk, secretions of the respiratory, intestinal and genitourinary tracts, and leukophoresis samples, or in tissue biopsies, swabs or smears.

Chromosomal deletions involving tumor suppressor genes may play an important role in the development and progression of solid tumors. The retinoblastoma tumor suppressor gene (Rb-1), located in chromosome 13q14, is the most extensively characterized tumor suppressor gene. Altered or lost expression of the Rb protein is caused by inactivation of both gene alleles either through a point mutation or a chromosomal deletion. Rb-i gene alterations have been found to be present not only in retinoblastomas but also in other malignancies such as osteosarcomas, small cell lung cancer, and breast cancer. Restriction fragment length polymorphism (RFLP) studies have indicated that such tumor types have frequently lost heterozygosity at 13q suggesting that one of the Rb-1 gene alleles has been lost due to a gross chromosomal deletion. Chromosome 1 abnormalities including duplications, deletions and unbalanced translocations involving chromosome 6 and other partner chromosomes indicate that regions of chromosome 1, in particular 1q21-1q32 and 1p11-13, might harbor oncogenes or tumor suppressor genes that are pathogenetically relevant to both chronic and advanced phases of myeloproliferative neoplasms. Myeloproliferative neoplasms are also associated with deletions of chromosome 5. Complete loss or interstitial deletions of chromosome 5 are the most common karyotypic abnormality in myelodysplastic syndromes (MDSs). Isolated del(5q)/5q-MDS patients have a more favorable prognosis than those with additional karyotypic defects, who tend to develop myeloproliferative neoplasms (MPNs) and acute myeloid leukemia. Further candidate microdeletions associated with cancer may include the ribosomal subunit RPS14, the transcription factor Egr1/Krox20 and the cytoskeletal remodeling protein, alpha-catenin. Cytogenetic and allelotyping studies of fresh tumours and tumour cell lines have shown that allelic loss from several distinct regions on chromosome 3p, including 3p25, 3p21-22, 3p21.3, 3p12-13 and 3p14, are the earliest and most frequent genomic abnormalities involved in a wide spectrum of major epithelial cancers of lung, breast, kidney, head and neck, ovary, cervix, colon, pancreas, esophagous, bladder and other organs. Several tumor suppressor genes have been mapped to the chromosome 3p region, and are thought that interstitial deletions or promoter hypermethylation precede the loss of the 3p or the entire chromosome 3 in the development of carcinomas.

Newborns and children with Down syndrome (DS) often present with congenital transient leukemia and have an increased risk of acute myeloid leukemia and acute lymphoblastic leukemia. Chromosome 21, harboring about 300 genes, may be involved in numerous structural aberrations, e.g., translocations, deletions, and amplifications, in leukemias, lymphomas, and solid tumors. Moreover, genes located on chromosome 21 have been identified that play an important role in tumorigenesis. Somatic numerical as well as structural chromosome 21 aberrations are associated with leukemias, and specific genes including RUNX1, TMPRSS2, and TFF, which are located in 21q, play a role in tumorigenesis.

In some embodiments, at least 5 microdeletions of interest are amplified in a single reaction volume, and wherein a construct for use as a positive control is prepared for each of the at least 5 microdeletions of interest. In some embodiments, the herein disclosed method of preparing a preparation of amplified DNA derived from a sample or a fraction thereof useful for identifying one or more microdeletions associated with a disease or disorder, and the one or more microdeletions comprise 22q11.2 deletion (DiGeorge syndrome), chromosome 5p15.2 (Cri-du-chat), 1p36 deletion, 15q11.2˜q13 deletion (Prader-Willi syndrome), and/or 15q11˜q13 (Angelman syndrome).

Further examples of microdeletion regions of interest include one or more of the regions associated with the following genetic conditions and diseases: 1q21.1 Distal Microdeletion, 2q37 Microdeletion: Albright Hereditary Osteodystrophy-like/Brachydactyly, 3q29 Microdeletion, Wolf-Hirschhorn syndrome, William-Beuren Syndrome, Langer-Giedion/Trichorhinophalangeal type II, 9q34 Microdeletion/Kleefstra Syndrome, 10p13-p14 DiGeorge 2, 11p13 Microdeletion: WAGR, 11q24.1 Microdeletion: Jacobsen Syndrome, Angelman Syndrome Type 2, Prader-Willi Syndrome Type 2, Prader-Willi, 16p11.2 Microdeletion, 16pter-p13.3 Microdeletion: AT-ID, Smith Magenis, Miller Dieker Syndrome, RCAD (17q12 del), 17q21.31 Microdeletion, 18q21.2 Microdeletion: Pitt-Hopkins Syndrome, DiGeorge, 22q11.21 Microdeletion, 22q11.2 Microdeletion, Phelan McDermid 22q13 Deletion, 5q22 Microdeletion: Familial Adenomatous Polyposis with ID, 5q35.2-35.3 Microdeletion-Sotos Syndrome, 6p25.3 (p24) Microdeletion, 8p23.1 Microdeletion CDH2, 11p11.2 Microdeletion: Potocki-Shaffer Syndrome, 13q14.2 Deletion, Retinoblastoma with ID, 13q32 Deletion-HPE5, PKD1/TSC2 Contiguous Deletion Syndrome, 17p13.3 Distal Microdeletion, 17p13.3 Distal Microdeletion, 17q21.31 Microdeletion, Isochromosome, 21q22.3 Microdeletion: Holoprosencephaly 1, Pelizaeus Merzbacher XL.

Microdeletions of interest may in some embodiments be associated with a cancer.

In some embodiments, the herein disclosed method of preparing a preparation of amplified DNA derived from a sample or a fraction thereof useful for identifying one or more microdeletions associated with a disease or disorder further comprises sequencing to detect (i) the presence of the construct as a positive control, and (ii) the presence of the one or more microdeletions of interest.

In some embodiments, the herein disclosed method of preparing a preparation of amplified DNA derived from a sample or a fraction thereof useful for identifying one or more microdeletions associated with a disease or disorder an efficiency and an error rate is determined for each amplification reaction by using the positive control, wherein the efficiency and the error rate is used to determine the presence of the one or more microdeletions of interest.

The microdeletions of interest may be identified by combining the positive control constructs disclosed herein with the methods for identifying microdeletions described in U.S. Ser. No. 17/252,205, incorporated by reference in its entirety.

In some embodiments, the present disclosure provides an amount of the construct to be added to the sample is determined by (a) mixing mono-nucleosomal DNA from normal female cell line and the construct in a range of proportions to generate a titration series to determine the Limit of Detection; (b) adding the mixture from (a) to DNA depleted plasma; (c) perform targeted amplification of the microdeletion that the construct is positive control for; and (d) determination of the proportion of the construct and the mono-nucleosomal DNA from the normal cell line that allows detection of the construct.

Some embodiments of the invention are kits comprising the composition comprising 2, 3, 4, 5, 6, 7, 8, 9, 10, 11, 12, 15, 20, 25 or 50 different engineered nucleic acid constructs on the low end and 3, 4, 5, 6, 7, 8, 9, 10, 11, 12, 15, 20, 25, 50, 100 or 250 different engineered nucleic acid constructs on the high end, wherein each construct is a positive control for a microdeletion region of interest. In some embodiments, the kit may comprise composition comprising (i) a first engineered nucleic acid construct comprising a barcode replacing a microdeletion of interest corresponding to 22q11.2 deletion, (ii) a second engineered nucleic acid construct comprising a barcode replacing a microdeletion of interest corresponding to 5p15.2 deletion, (iii) a third engineered nucleic acid construct comprising a barcode replacing a microdeletion of interest corresponding to 1p36 deletion, (iv) a fourth engineered nucleic acid construct comprising a barcode replacing a microdeletion of interest corresponding to 15q11.2-q13 deletion, and/or (v) a fifth engineered nucleic acid construct comprising a barcode replacing a microdeletion of interest corresponding to 15q11-q13 deletion. The kit may comprise primers for amplification of one or microdeletions of interest and the corresponding engineered nucleic acid constructs to be used as a positive control.

### Isolation of Nucleic Acids

The term “isolating” as used herein refers to a physical separation of the target genetic material from other biological material. It may also refer to a partial isolation, where the target of isolation is separated from some or most, but not all of the biological material.

Isolation of mononucleosomal DNA: It has been shown that cfDNA may exist as nucleosomal complexes with the DNA tightly wrapped around histones. Mononucleosomal complexes consists of about 130 to about 170 bp of DNA wrapped around a single nucleosome. The term “mononucleosomal” refers to a fragment of chromosomal DNA containing a single nucleosome or to a nucleic acid sequence comprising the size of a mononucleosomal DNA, which is from about 130 to about 170 bp. The term “sub-mononucleosomal” refers to a fragment of chromosomal DNA having smaller molecular size than about 130 bp that would be expected to derive from a complete nucleosome. cfDNA may also exist integrated in lipid vesicles such as exosomes.

Chromosomal DNA consists of DNA wrapped around a complex of histone proteins that forms a nucleosome. The nucleosome protects the DNA so that fragmented chromosomal DNA are often found as multiples of nucleosomes.

Many methods known by a person of ordinary skill in the art may be used to isolate cell-free DNA from a biological sample. Such methods include but are not limited to organic liquid phase extraction utilizing phenol and phenol-chloroform mixtures to disintegrate nucleoprotein complexes and sequester proteins and lipids into the organic phase while partitioning the highly hydrophilic DNA and RNA into the aqueous phase in very pure form. Other methods include using agarose hydrogels such as those described in E. M. Southern (J. Mol. Biol. (1975) 94:51-70) and Vogelstein and Gillespie (PNAS, USA (1979)76:615-619), incorporated herein in their entirety. Another method is to capture DNA on a solid phase material as described in Boom et al. (J Clin Micro. (1990) 28(3):495-503), incorporated herein in its entirety. Methods for DNA isolation in general can be found in Sambrook J, Russel D W (2001). Molecular Cloning: A Laboratory Manual 3rd Ed. Cold Spring Harbor Laboratory Press. Cold Spring Harbor, NY, incorporated herein.

In a particular aspect, the cfDNA is extracted by using an ion exchange based beads method.

Further methods described in detail below can be used to enrich for DNA fragments within specific molecular size ranges.

### Size Selection/Exclusion Methods

This disclosure relates to methods comprising performing size selection by gel electrophoresis, paramagnetic beads, spin column, salt precipitation, or biased amplification.

In some embodiments, the size exclusion step of the methods disclosed herein is performed by using gel electrophoresis to separate the cfDNA samples according to size and selecting a determined size range. Gel electrophoresis is an art-recognized method for separating DNA molecules based on their size by applying an electric field to a gel, such as an agarose gel, upon which DNA molecules will move through the gel towards the positively charged anode. The size of the DNA molecules will determine the speed by which the DNA molecule migrate through the gel. A standard mixture of DNA molecules with predetermined sizes can be applied to the gel to identify the size of the DNA. The DNA molecules of desired size can then be extracted and purified by using well-known techniques such as those disclosed in Sambrook J, Russel D W (2001). Molecular Cloning: A Laboratory Manual 3rd Ed. Cold Spring Harbor Laboratory Press. Cold Spring Harbor, NY, incorporated. In some embodiments, the size selection is performed on an automated high-throughput gel electrophoresis system such as Pippin or Costal Genomics systems.

In some embodiments, the size exclusion step of the methods disclosed herein is performed by using paramagnetic beads. The use of paramagnetic beads for size selection of DNA fragments is described in DeAngelis et al., Solid-Phase Reversible Immobilization for the Isolation of PCR Products, Nucleic Acid Research, November 23(22): 4742-3 (1995), incorporated herein. In brief, this method is based on that DNA fragment size affects the total charge per molecule with larger DNAs having larger charges, which promotes their electrostatic interaction with the beads and displaces smaller DNA fragments. Thus, by manipulating the composition of the buffer solution used to mix beads and DNA, the beads can be made to bind DNA within specific size ranges. The most famous and highly applied approach is Solid Phase Reversible Isolation (SPRI) selection which utilizes carboxyl coated paramagnetic beads in the presence of high salt and the crowding agent polyethylene glycol (PEG), to promote controlled adsorption, configure to bind DNA molecules within a certain molecular weight ranges by varying PEG concentrations. DNA molecules of differing length can be partitioned by subjecting source DNA to various binding and elution schemes in the presence of different amounts of PEG. In some embodiments, AMPURE™ beads are used for the size exclusion step.

In some embodiments, the size exclusion step of the methods disclosed herein is performed by using spin columns. A spin column contains material that will absorb molecules based on the size of the molecules. The spin column material contains pores of defined sizes and molecules with a size above a cutoff size determined by the pore size will not enter the pores, and are eluted with the column's void volume. Different types of column material can be chosen to achieve absorption or exclusion of DNA molecules within various size ranges. In some embodiments, the spin column material comprises siliceous materials, silica gel, glass, glass fiber, zeolite, aluminum oxide, titanium dioxide, zirconium dioxide, kaolin, gelatinous silica, magnetic particles, ceramics, polymeric supporting materials, or a combination thereof. In a particular embodiment, the spin column material comprises glass fiber.

In some embodiments, spin columns may be used for size exclusion by using different binding buffers configured to provide low or high stringency binding conditions when applying the DNA samples to the spin column, as described in PCT patent application No. PCT/US2019/18274 filed on Feb. 15, 2019, which is incorporated herein by reference in its entirety. Under low stringency binding conditions, the spin column material be configured to restrict binding of DNA fragments of low molecular weights, whereas high stringency binding conditions will configure the spin column to facilitate binding of DNA fragments with low molecular weights.

In some embodiments, the low and/or high stringency binding buffer comprises a nitrile compound selected from acetonitrile (ACN), propionitrile (PCN), butyronitrile (BCN), isobutylnitrile (IBCN), or a combination thereof. The first and/or second binding buffer can comprise, for example, about 15% to about 35%, or about 20% to about 30%, or about 25% of the nitrile compound (e.g., ACN).

In some embodiments, the low and/or high stringency binding buffer comprises a chaotropic compound selected from GnCl, urea, thiourea, guanidine thiocyanate, Nal, guanidine isothiocyanate, D-/L-arginine, a perchlorate or perchlorate salt of Li+, Na+, K+, or a combination thereof. The low and/or high stringency binding buffer can comprise, for example, about 5 M to about 8 M, or about 5.6 M to about 7.2 M, or about 6 M of the chaotropic compound (e.g., GnCl).

The binding buffers may also comprise an alcohol, a chelating agent, and a detergent. In some embodiments, the alcohol is propanol. In some embodiments, the chelating compound comprises ethylenediaminetetraccetic (EDTA), ethyleneglycol-bis(2-aminoethylether)-N,N,N′,N′-tetraacetic acid (EGTA), citric acid, N,N,N′,N′-Tetrakis(2-pyridylmethyl)ethylenediamine (TPEN), 2,2′-Bipyridyl, deferoxamine methanesulfonate salt (DFOM), 2,3-Dihydroxybutanedioic acid (tartaric acid), or a combination thereof. In some embodiments, the detergent may be Triton X-100, Tween 20, N-lauroyl sarcosine, sodium dodecylsulfate (SDS), dodecyldimethylphosphine oxide, sorbitan monopalmitate, decylhexaglycol, 4-nonylphenyl-polyethylene glycol, or a combination thereof. In a particular embodiment, the detergent is Triton X-100.

In some embodiments, the size exclusion step of the methods disclosed herein is performed by using salt precipitation. Larger DNA molecules will precipitate at lower salt concentrations than smaller DNA molecules. By varying the concentration of salt in the precipitation buffer, DNA molecules in different size ranges can be separated.

### Amplification Methods

In some embodiments, the method comprises performing targeted amplification to identify the microdeletions of interest. In some embodiments, the targeted amplification to identify the microdeletions of interest comprises performing amplification of a plurality of amplification reactions. In some embodiments, the amplification reactions are single reactions performed in parallel. In some embodiments, the target loci are amplified by performing multiplex amplification of a plurality of target loci in a single reaction. In some embodiments, the amplification reactions to identify the microdeletions of interest are performed by using multiplex amplification of a plurality of target loci in a single reaction. In some embodiments, the at least 5 microdeletions of interest are identified by performing a multiplex amplification in a single reaction volume.

In some embodiments, the reaction mixture for performing multiplex amplification comprises the engineered construct for use as positive control for identifying the microdeletions of interest.

Multiplex amplification reactions can be set up as a single reaction or as pools of different subset multiplex reactions. The multiplex reaction methods provided herein, such as the massive multiplex PCR disclosed herein provide an exemplary process for carrying out the amplification reaction to help attain improved multiplexing and therefore, sensitivity levels.

In certain illustrative embodiments, the nucleic acid sequence data is generated by performing high throughput DNA sequencing of a plurality of copies of a series of amplicons generated using a multiplex amplification reaction, wherein each amplicon of the series of amplicons spans at least one polymorphic loci of the set of polymorphic loci and wherein each of the polymeric loci of the set is amplified. For example, in these embodiments a multiplex PCR to amplify amplicons across the 5 microdeletion regions may be performed, and wherein the amplicons comprise a plurality of polymorphic loci.

In some embodiments, amplification is performed using direct multiplexed PCR, sequential PCR, nested PCR, doubly nested PCR, one-and-a-half sided nested PCR, fully nested PCR, one sided fully nested PCR, one-sided nested PCR, hemi-nested PCR, hemi-nested PCR, triply hemi-nested PCR, semi-nested PCR, one sided semi-nested PCR, reverse semi-nested PCR method, or one-sided PCR, which are described in U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, and U.S. Ser. No. 61/994,791, filed May 16, 2014, which are hereby incorporated by reference in their entirety.

In an embodiment, a multiplex PCR assay is designed to amplify microdeletion regions of interest and/or to amplify a SNP within or flanking the microdeletion of interest.

In some embodiments, the method of amplifying target loci in a nucleic acid sample involves (i) contacting the nucleic acid sample with a library of primers that simultaneously hybridize to least 5, 10, 20, 30, 40, 50, 60, 70, 80, 90 100; 200; 500; 750; 1,000; 2,000; 5,000; 7,500; 10,000; 20,000; 25,000; 30,000; 40,000; 50,000; 75,000; or 100,000 different target loci to produce a single reaction mixture; and (ii) subjecting the reaction mixture to primer extension reaction conditions (such as PCR conditions) to produce amplified products that include target amplicons. In some embodiments, at least 50, 60, 70, 80, 90, 95, 96, 97, 98, 99, or 99.5% of the targeted loci are amplified. In various embodiments, less than 60, 50, 40, 30, 20, 10, 5, 4, 3, 2, 1, 0.5, 0.25, 0.1, or 0.05% of the amplified products are primer dimers. In some embodiments, the primers are in solution (such as being dissolved in the liquid phase rather than in a solid phase). In some embodiments, the primers are in solution and are not immobilized on a solid support. In some embodiments, the primers are not part of a microarray.

In certain embodiments, the multiplex amplification reaction is performed under limiting primer conditions for at least ½ of the reactions. In some embodiments, limiting primer concentrations are used in 1/10, ⅕, ¼, ⅓, ½, or all of the reactions of the multiplex reaction. Provided herein are factors to consider to achieve limiting primer conditions in an amplification reaction such as PCR.

The PCR reaction conditions may be for example the STAR (Specific Target Amplification Reaction) protocol disclosed in U.S. Ser. No. 17/545,881, incorporated herein in its entirety. For example, the PCR reaction can be performed with an annealing temperature between 1, 2, 3, 4, 5, 6, 7, 8, 9, or 10° C. greater than the melting temperature on the low end of the range, and 2, 3, 4, 5, 6, 7, 8, 9, 10, 11, 12, 13, 14 or 15° on the high end the range for at least 10, 20, 25, 30, 40, 50, 06, 70, 75, 80, 90, 95 or 100% the primers of the set of primers.

In certain embodiments, wherein the amplification reaction is a PCR reaction the length of the annealing step in the PCR reaction is between 1, 10, 15, 20, 30, 45, and 60 minutes on the low end of the range, and 15, 20, 30, 45, 60, 120, 180, or 240 minutes on the high end of the range. In certain embodiments, the primer concentration in the amplification, such as the PCR reaction is between 1 and 10 nM. Furthermore, in exemplary embodiments, the primers in the set of primers, are designed to minimize primer dimer formation.

Accordingly, in an example of any of the methods herein that include an amplification step, the amplification reaction is a PCR reaction, the annealing temperature is between 1 and 10° C. greater than the melting temperature of at least 90% of the primers of the set of primers, the length of the annealing step in the PCR reaction is between 15 and 60 minutes, the primer concentration in the amplification reaction is between 1 and 10 nM, and the primers in the set of primers, are designed to minimize primer dimer formation. In a further aspect of this example, the multiplex amplification reaction is performed under limiting primer conditions.

In some embodiments, the following ranges of multiplex reactions are performed: between 1, 2, 3, 4, 5, 6, 7, 8, 9, 10, 20, 30, 40, 50, 60, 70, 80, 90, 100, 200, 250, 500, 1000, 2500, 5000, 10,000, 20,000, 25000, 50000 on the low end of the range and between 1, 2, 3, 4, 5, 6, 7, 8, 9, 10, 20, 30, 40, 50, 60, 70, 80, 90, 100, 200, 250, 500, 1000, 2500, 5000, 10,000, 20,000, 25000, 50000, and 100,000 on the high end of the range.

In an embodiment, a multiplex PCR assay is designed to amplify a SNP within or flanking the microdeletion of interest and these assays are used in a single reaction to amplify DNA. The number of PCR assays may be between 1 and 10, 5 and 10, 10 and 50, 50 and 200 PCR assays, between 5 and 20 PCR assays, between 100 and 1,000 PCR assays, between 1,000 and 10,000, or between 1,000 and 20,000, or more than 20,000 PCR assays (5 to 20-plex, 100 to 1,000-plex, 1,00 to 5,000-plex, 1,000 to 10,000-plex, 1,000 to 20,000-plex, or more than 20,000-plex respectively). In an embodiment, a multiplex pool of about 10,000 PCR assays (10,000-plex) are designed to amplify target loci of interest and these assays are used in a single reaction to amplify cfDNA obtained from a material plasma sample, chorion villus samples, amniocentesis samples, single or a small number of cells, other bodily fluids or tissues, cancers, or other genetic matter.

The SNP frequencies of each locus may be determined by clonal or some other method of sequencing of the amplicons. Statistical analysis of the allele frequency distributions or ratios of all assays may be used to determine if the sample contains a trisomy of one or more of the chromosomes included in the test. In another embodiment the original cfDNA samples is split into two samples and parallel 5,000-plex assays are performed. In another embodiment the original cfDNA samples is split into n samples and parallel (˜10,000/n)-plex assays are performed where n is between 2 and 12, or between 12 and 24, or between 24 and 48, or between 48 and 96.

Bioinformatics methods are used to analyze the genetic data obtained from multiplex PCR. The bioinformatics methods useful and relevant to the methods disclosed herein can be found in U.S. Patent Publication No. 20180025109, incorporated by reference herein.

### Hybrid Capture Methods

In some embodiments, the method comprises performing hybrid capture to select a plurality of polymorphic loci on the selectively enriched DNA before determining the sequences of the selectively enriched DNA.

In some embodiments, preferentially enriching the DNA at the plurality of polymorphic loci includes obtaining a plurality of hybrid capture probes that target the polymorphic loci, hybridizing the hybrid capture probes to the DNA in the sample and physically removing some or all of the unhybridized DNA from the first sample of DNA.

In some embodiments, the hybrid capture probes are designed to hybridize to a region that is flanking but not overlapping the polymorphic site. In some embodiments, the hybrid capture probes are designed to hybridize to a region that is flanking but not overlapping the polymorphic site, and where the length of the flanking capture probe may be selected from the group consisting of less than about 120 bases, less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases. In some embodiments, the hybrid capture probes are designed to hybridize to a region that overlaps the polymorphic site, and where the plurality of hybrid capture probes comprise at least two hybrid capture probes for each polymorphic loci, and where each hybrid capture probe is designed to be complementary to a different allele at that polymorphic locus.

### Nucleic Acid Sequencing

The sample nucleic acids can be further analyzed by genotyping microarrays, and high throughput sequencing. Some high throughput sequencing methods include Sanger DNA sequencing, pyrosequencing, the ILLUMINA SOLEXA platform, ILLUMINA's GENOME ANALYZER, or APPLIED BIOSYSTEM's 454 sequencing platform, HELICOS's TRUE SINGLE MOLECULE SEQUENCING platform, HALCYON MOLECULAR's electron microscope sequencing method, or any other sequencing method. In some embodiments, the high throughput sequencing is performed on Illumina NextSeq. In some embodiments, the high throughput sequencing methods comprise NovaSeq, BGI/MGI sequencing system, Omniome/PacBio sequencing system, Oxford Nanopore sequencing system, or Ion Torrent sequencing system.

In some embodiments, the sequences of the selectively enriched DNA are determined by performing microarray analysis. In an embodiment, the microarray may be an ILLUMINA SNP microarray, or an AFFYMETRIX SNP microarray.

In some embodiments, the sequences are determined by performing quantitative PCR (qPCR) or digital droplet PCR (ddPCR) analysis. qPCR measures the intensity of fluorescence at specific times (generally after every amplification cycle) to determine the relative amount of target molecule (DNA). ddPCR measures the actual number of molecules (target DNA) as each molecule is in one droplet, thus making it a discrete “digital” measurement. It provides absolute quantification because ddPCR measures the positive fraction of samples, which is the number of droplets that are fluorescing due to proper amplification. This positive fraction accurately indicates the initial amount of template nucleic acid.

As used herein, the term ‘adaptors,’ or ‘ligation adaptors’ or ‘library tags’ are DNA molecules containing a universal priming sequence that can be covalently linked to the 5-prime and 3-prime end of a population of target double stranded DNA molecules. In some embodiments, the addition of the adapters provides universal priming sequences to the 5-prime and 3-prime end of the target population from which PCR amplification can take place, amplifying all molecules from the target population, using a single pair of amplification primers. Disclosed herein are methods that permit the targeted amplification of over a hundred to tens of thousands of target sequences (e.g. SNP loci) from genomic DNA obtained from plasma. The amplified sample may be relatively free of primer dimer products and have low allelic bias at target loci. If during or after amplification the products are appended with sequencing compatible adaptors, analysis of these products can be performed by sequencing. These methods are more fully described in U.S. Patent Publications 20170242960 and 20180025109, and U.S. Pat. No. 9,163,282, incorporated herein.

In some embodiments, the adaptors or primers describe herein may comprise one or more molecular barcodes. Molecular barcodes or molecular indexing sequences may be used in next generation sequencing to reduce quantitative bias introduced by replication. In next generation sequencing, each nucleic acid fragment may be tagged with a molecular barcode or molecular indexing sequence. Sequence reads that have different molecular barcodes or molecular indexing sequences represent different original nucleic acid molecules. By referencing the molecular barcodes or molecular indexing sequences, PCR artifacts, such as sequence changes generated by polymerase errors that are not present in the original nucleic acid molecules can be identified and separated from real variants/mutations present in the original nucleic acid molecules.

In some embodiments, molecular barcodes are introduced by ligating adaptors carrying the molecular barcodes to the isolated cfDNA to obtain adaptor-ligated and molecular barcoded DNA. In some embodiments, molecular barcodes are introduced by amplifying the adaptor-ligated DNA with primers carrying the molecular barcodes to obtain amplified adaptor-ligated and molecular barcoded DNA.

In some embodiments, the molecular barcoding adaptor or primers may comprise a universal sequence, followed by a molecular barcode region, optionally followed by a target specific sequence in the case of a primer. The sequence 5′ of molecular barcode may be used for subsequence PCR amplification or sequencing and may comprise sequences useful in the conversion of the amplicon to a library for sequencing. The random molecular barcode sequence could be generated in a multitude of ways. The preferred method synthesizes the molecule tagging adaptor or primer in such a way as to include all four bases to the reaction during synthesis of the barcode region. All or various combinations of bases may be specified using the IUPAC DNA ambiguity codes. In this manner the synthesized collection of molecules will contain a random mixture of sequences in the molecular barcode region. The length of the barcode region will determine how many adaptors or primers will contain unique barcodes. The number of unique sequences is related to the length of the barcode region as NL where N is the number of bases, typically 4, and L is the length of the barcode. A barcode of five bases can yield up to 1024 unique sequences; a barcode of eight bases can yield 65536 unique barcodes. In an embodiment, the DNA can be measured by a sequencing method, where the sequence data represents the sequence of a single molecule. This can include methods in which single molecules are sequenced directly or methods in which single molecules are amplified to form clones detectable by the sequence instrument, but that still represent single molecules, herein called clonal sequencing.

In some embodiments, the molecular barcodes described herein are Molecular Index Tags (“MITs”), which are attached to a population of nucleic acid molecules from a sample to identify individual sample nucleic acid molecules from the population of nucleic acid molecules (i.e. members of the population) after sample processing for a sequencing reaction. MITs are described in detail in U.S. Pat. No. 10,011,870 to Zimmermann et al., which is incorporated herein by reference in its entirety. Unlike prior art methods that relate to unique identifiers and teach having a diversity of unique identifiers that is greater than the number of sample nucleic acid molecules in a sample in order to tag each sample nucleic acid molecule with a unique identifier, the present disclosure typically involves many more sample nucleic acid molecules than the diversity of MITs in a set of MITs. In fact, methods and compositions herein can include more than 1,000, 1×10⁶, 1×10⁹, or even more starting molecules for each different MIT in a set of MITs. Yet the methods can still identify individual sample nucleic acid molecules that give rise to a tagged nucleic acid molecule after amplification.

### General Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

### WORKING EXAMPLES

### Example 1

This example shows construction of an engineered construct for use as a positive control in methods of identifying a microdeletion of interest, and use of the construct for identification of microdeletions of interest.

The overall method for construction of an engineered construct for use as a positive control in methods of identifying a microdeletion of interest is shown in FIG. 1. First, a reference DNA sequence is obtained that contains a microdeletion region of interest. In FIG. 1 the “^” symbol indicates a SNP, and the “v” indicates an inverted SNP. Second, a region within the microdeletion region is deleted and replaced with a barcode of 6 to 9 base pairs. The 5′ and 3′ flanking regions of the central microdeletion region comprise binding sites for primers suitable for use with for example STAR (Specific Target Amplification Reaction) primers targeting microdeletions. The primers may be designed for identification of 22q11.2 deletion (DiGeorge syndrome), chromosome 5p15.2 (Cri-du-chat), 1p36 deletion, 15q11.2˜q13 deletion (Prader-Willi syndrome), and/or 15q11˜q13 (Angelman syndrome).

The construct is prepared by using chemical synthesis and subsequent PCR amplification of the synthesized construct.

The engineered construct for use as a positive control was mixed with a sample of nucleic acids from a subject to obtain a spiked sample. The amount of the construct to be added to the sample is determined by (a) mixing mono-nucleosomal DNA from normal female cell line and the construct in a range of proportions to generate a titration series to determine the Limit of Detection; (b) adding the mixture from (a) to DNA depleted plasma; (c) perform targeted amplification of the microdeletion that the construct is positive control for; and (d) determination of the proportion of the construct and the mono-nucleosomal DNA from the normal cell line that allows detection of the construct. The identified amount of the construct is added to the sample from the subject and targeted amplification of the microdeletions of interest is performed. The construct will be amplified as a positive control and can be identified by the barcodes that replaced the microdeletion region. The microdeletions of interest may be amplified in a single reaction volume, in which case a composition comprising positive control construct for all microdeletions of interests are added to the sample. The amplification of the positive control constructs allows for determination of an efficiency and an error rate that can be used to calibrate the results of amplification of the sample nucleic acids to improve the accuracy of identification of the microdeletions of interest.

The demonstration that the SNP assays flanking the barcodes that replace the microdeletion in the engineered construct work in the STAR reactions indicates that the assays flanking and within the deletion work. Thus, the constructs can be used as positive controls.

What is claimed is:

1. A composition comprising an engineered nucleic acid construct for use as a positive control for one or more primers targeting one or more microdeletions of interest in a sample, wherein the construct is engineered from a reference nucleic acid sequence comprising a microdeletion of interest, wherein the construct comprises a 5′ end region, a central region, and a 3′ end region, wherein the 5′ end region and 3′ end region comprise reference sequences flanking the microdeletion of interest, wherein the central region of the construct is a DNA barcode, and wherein the DNA barcode replaces the microdeletion sequence of the reference nucleic acid sequence.

2. The composition of claim 1, wherein the microdeletion of interest corresponds to 22q11.2 deletion, 5p15.2 deletion, 1p36 deletion, 15q11.2-q13 deletion, or 15q11-q13 deletion.

3. The composition of claim 1, wherein the composition comprises (i) a first engineered nucleic acid construct comprising a barcode replacing a microdeletion of interest corresponding to 22q11.2 deletion, (ii) a second engineered nucleic acid construct comprising a barcode replacing a microdeletion of interest corresponding to 5p15.2 deletion, (iii) a third engineered nucleic acid construct comprising a barcode replacing a microdeletion of interest corresponding to 1p36 deletion, (iv) a fourth engineered nucleic acid construct comprising a barcode replacing a microdeletion of interest corresponding to 15q11.2-q13 deletion, and (v) a fifth engineered nucleic acid construct comprising a barcode replacing a microdeletion of interest corresponding to 15q11-q13 deletion.

4. The composition of claim 1, wherein the microdeletion of interest is associated with a cancer.

5. The composition of claim 1, wherein the barcode is from about 6 base pairs to about 9 base pairs.

6. The composition of claim 1, wherein the 5′ and 3′ end regions of the construct comprise at least one single nucleotide polymorphism (SNP) of interest.

7. The composition of claim 1, wherein the 5′ and 3′ end regions of the construct comprise sequences recognized by primers that target a SNP within or flanking the microdeletion of interest.

8. The composition of claim 3, wherein the reference nucleic acid sequence is maternal DNA, and wherein the SNP of interest is changed to allow the construct to act as the positive control for a child DNA.

9. The composition of claim 1, wherein the size of the construct is from about 100 bp to about 200 bp, or from about 160 bp to about 200 bp.

10. A method of preparing the construct for use as a positive control for one or more primers targeting one or more microdeletions of interest in a sample according to claims 1-9, wherein the method comprises obtaining a reference nucleic acid; isolating a nucleic acid sequence comprising a 5′ end and 3′ end flanks a microdeletion of interest; and replacing the central region of the reference nucleic acid sequence corresponding to the microdeletion of interest with a barcode.

11. The method of claim 10, wherein the preparing a construct for use as a positive control for one or more primers targeting one or more microdeletions is performed by chemical synthesis of the construct and subsequent PCR amplification of the synthesized construct.

12. The method of claim 10, wherein the sample is a plasma sample and comprises cell-free DNA.

13. The method of claim 10, wherein the sample comprises circulating tumor DNA (ctDNA).

14. The method of claim 10, wherein the sample comprises cells and/or tissues.

15. The method of claim 10, wherein the reference nucleic acid is obtained from a cell line suitable for use as a positive control for detecting the one or more microdeletions.

16. The method of claim 10, wherein the reference nucleic acid is genomic DNA.

17. The method of claim 10, wherein the reference nucleic acid is mono-nucleosomal.

18. A method of preparing a preparation of amplified DNA derived from a sample or a fraction thereof useful for identifying one or more microdeletions associated with a disease or disorder, comprising:
(a) preparing a construct for use as a positive control for detection of one or more microdeletions according to claims 10-17;
(b) adding the construct from (a) into the sample or fraction thereof to obtain a spiked sample and extracting nucleic acids from the spiked sample or fraction thereof;
(c) performing targeted amplification on the spiked sample or fractions thereof from (b) to amplify one or more target regions comprising microdeletions of interests to obtain amplicons; and
(d) analyzing the amplicons or portions thereof from (c) to determine (i) whether the amplicons comprises the amplified construct as a positive control, and (ii) whether the amplicons comprises the one or more microdeletions of interest.

19. The method of claim 18, wherein the sample is a plasma sample and comprises cell-free DNA.

20. The method of claim 19, wherein the plasma sample comprises maternal and fetal cell-free DNA, and wherein a SNP in the construct is changed to act as a positive control for the fetal cell-free DNA.

21. The method of claim 20, wherein at least 5 microdeletions of interest are amplified in a single reaction volume, and wherein a construct for use as a positive control is prepared for each of the at least 5 microdeletions of interest.

22. The method of claims 18-21, wherein the one or more microdeletions comprise 22q11.2 deletion (DiGeorge syndrome), chromosome 5p15.2 (Cri-du-chat), 1p36 deletion, 15q11.2˜q13 deletion (Prader-Willi syndrome), and/or 15q11˜q13 (Angelman syndrome).

23. The method of claims 18-21, wherein the one or more microdeletions are associated with a cancer.

24. The method of claim 18, further comprising sequencing to detect (i) the presence of the construct as a positive control, and (ii) the presence of the one or more microdeletions of interest.

25. The method of claim 18, wherein an efficiency and an error rate is determined for each amplification reaction by using the positive control, wherein the efficiency and the error rate is used to determine the presence of the one or more microdeletions of interest.

26. The method of claim 18, wherein an amount of the construct to be added to the sample is determined by (a) mixing DNA from normal female cell line and the construct in a range of proportions to generate a titration series to determine the Limit of Detection; (b) adding the mixture from (a) to DNA depleted plasma; (c) perform targeted amplification of the microdeletion that the construct is positive control for; and (d) determination of the proportion of the construct and the mono-nucleosomal DNA from the normal cell line that allows detection of the construct.

27. The method of claim 26, wherein the DNA is mono-nucleosomal DNA.

28. The method of claim 18, wherein the sample is a plasma sample from a mother.

29. A method of preparing a sample comprising nucleic acids, comprising spiking a sample with the composition according to claims 1-9.

30. The method according to claim 29, wherein the sample is a plasma sample from a mother.

31. The method of any one of claims 18-30, wherein detection of SNPs flanking the barcodes that replace the microdeletion in the engineered construct in the amplicons demonstrates that the assays for detecting SNPs flanking and within the microdeletion work, thereby confirming that the engineered constructs can be used as the positive control.
